(19) United States
(12) Patent Application Publication
(10) Pub. No.: US 2021/0252466 A1
(43) Pub. Date: Aug. 19, 2021

(54) BAFFLE INSERT DEVICE AND METHOD OF USE

(71) Applicant: CORNING INCORPORATED, Corning, NY (US)

(72) Inventor: Ryann Loren Russell, Drexel Hill, PA (US)

(21) Appl. No.: 16/973,967

(22) PCT Filed: Jun. 4, 2019
(86) PCT No.: PCT/US2019/035297

§ 371 Date: Dec. 10, 2020

Related U.S. Application Data
(60) Provisional application No. 62/684,928, filed on Jun. 14, 2018.

(51) Int. Cl. B01F 15/00; C12M 1/00

(52) U.S. Cl. CPC B01F 15/00896; C12M 27/20

(57) ABSTRACT

A removable baffle insert can be used in connection with containers such as bioreactors. The baffle insert can include a plurality of elongated baffle members configured to fit in a tube and contact the interior wall of the tube. The baffle insert can also include interconnection members coupled to the plurality of elongated baffle members. The interconnection members hold the plurality of baffle members together in a spaced apart relationship. In some embodiments, the baffle insert has a cross-sectional geometry that is larger than a cross-sectional geometry of the tube and the interconnection member is configured to bias the plurality of baffle members against the interior wall of the tube. In some embodiments, the baffle insert can be held in place by a cap attached to the container

### CROSS-REFERENCE TO RELATED APPLICATION

This application claims the benefit of priority under 35 U.S.C. § 120 of U.S. Provisional Application Ser. No. 62/684928 filed on Jun. 14, 2018, the content of which is relied upon and incorporated herein by references in its entirety.

### FIELD

The following relates generally to baffle inserts configured to fit within a tube, and more specifically to a removable baffle insert for use within a bioreactor.

### BACKGROUND

Bioprocesses often take place in bioreactors. Bioreactors may be smooth walled containers (e.g., tubes) designed for growing cultures in an environment where the system inputs and/or outputs are controlled. For example, the oxygen input into a bioreactor may be an important factor for culture growth and may be controlled using a vent in the bioreactor. Bioreactors may be agitated to increase mixing in the culture and allow more oxygen to enter the system. Bioreactors may be used for high throughput suspension culture processes. Exemplary applications of high throughput bioreactors may include cell line development, clone selection, media optimization, recombinant protein development, etc.

Cultures in a bioreactor often require adequate oxygen to enable biomass growth. When a culture's oxygen uptake rate exceeds the system's oxygen transfer rate, the culture system is in an oxygen deficit. Cell cultures and microbial applications in bioreactors may require high amounts of oxygen for proper growth. Conventional high throughput environments and other small volume cultures in a smooth walled bioreactor may have an oxygen deficit. Also, small scale applications in bioreactors may be more likely to have an oxygen deficit than large scale applications. Dynamic gassing may be used to increase an oxygen uptake rate in a bioreactor, but the controls for dynamic gassing become complicated when deployed in high density cultures, such as tubes.

### SUMMARY

The present disclosure is directed towards a removable baffle insert that can be used in a container such as a bioreactor. In some embodiments, the removable baffle insert is designed to be inserted into a container such that the baffles exert a biasing force on the interior wall of the container. Thus, the baffle insert may be temporarily secured to the interior wall of the container, including when the container is inverted, used to grow a culture, undergoes a mixing process, and the like. This may be achieved by the baffle insert having a cross-sectional geometry that is larger than the cross-sectional geometry of the container.

In some embodiments, the container is a bioreactor including a tube and the removable baffle insert. The tube has an opening at a top end through which the removable baffle insert can be positioned in the tube. The tube can also be closed at a bottom end. The bioreactor may also include a cap coupled to the opening of the tube. The bioreactor may be configured to facilitate the growth of cell cultures, microorganisms, and the like in the tube and on the baffle insert.

The baffle insert may include a plurality of elongated baffle members coupled together by one or more interconnection members. The interconnection member may hold the plurality of elongated baffle members together in a symmetrical, spaced apart relationship. For example, the interconnection member can be a ring and the plurality of elongated baffle members can be evenly dispersed around the ring.

The plurality of elongated baffle members can be positioned parallel to the longitudinal axis of the bioreactor (e.g., a tube). In some embodiments, the plurality of elongated baffle members may be configured to fit in the tube and contact an interior wall of the tube. The baffle insert may have a cross-sectional geometry that is larger than a cross-sectional geometry of the tube—e.g., the baffle insert can have a diameter that is larger than a diameter of the tube. The baffle insert can be positioned in the tube by contracting it, placing it in the tube, and allowing it to expand and contact the interior wall of the tube.

The interconnection member can be flexible and arranged perpendicular to the plurality of baffle members. The interconnection member may be configured to move between a relaxed state when the baffle insert is not positioned in the tube and a flexed state when the baffle insert is positioned in the tube and the interconnection member biases the plurality of baffle members against the interior wall of the tube. The interconnection member can include a flexible band and/or a crossbar.

The container and the baffle insert can be made of any suitable material. In some embodiments, the container and/or the baffle insert are made of a polymer such as a polyolefin, glass, and the like.

The bioreactor can also have a variety of shapes. In some embodiments, the bioreactor has a conical or sloped bottom and the plurality of baffle members include sloped or angled bottoms that correspond to the interior shape of the bioreactor. In some examples, the bioreactor may include graduation markings on the exterior of the bioreactor. In some embodiments, the bioreactor comprises a culture of cells and/or microorganisms in the tube.

In some embodiments, after the baffle insert is temporarily secured to the interior tube wall, the baffle insert may be removed allowing for further processing of the tube contents. For example, a tube may undergo a first process without the baffle insert, a user may then place the baffle insert into the tube for a second process, and, finally, the user may remove the baffle insert after the second process so that the tube may undergo a third process. Thus, the removable baffle insert provides greater flexibility for the use of a single bioreactor.

One or more representative embodiments is provided to illustrate the various features, characteristics, and advantages of the disclosed subject matter. The embodiments are provided in the context of a bioreactor. It should be understood, however, that many of the concepts can be used in a variety of other settings, situations, and configurations. For example, the features, characteristics, advantages, etc., of one embodiment can be used alone or in various combinations and sub-combinations with one another.

The summary is provided to introduce a selection of concepts in a simplified form that are further described below in the detailed description. The summary and the background are not intended to identify key concepts or essential aspects of the disclosed subject matter, nor should they be used to constrict or limit the scope of the claims. For example, the scope of the claims should not be limited based on whether the recited subject matter includes any or all aspects noted in the summary and/or addresses any of the issues noted in the background.

### DRAWINGS

The preferred and other embodiments are disclosed in association with the accompanying drawings in which:

FIG. 1 is a perspective view of a baffle insert within a tube in accordance with embodiments of the present disclosure.

FIG. 2 is a perspective view of a baffle insert within a tube in accordance with embodiments of the present disclosure.

FIG. 3 is a perspective view of a baffle insert in accordance with embodiments of the present disclosure.

FIG. 4 is a top view of a baffle insert in accordance with embodiments of the present disclosure.

FIG. 5 is a top view of a baffle insert in accordance with embodiments of the present disclosure.

FIG. 6 is a top view of a baffle insert in accordance with embodiments of the present disclosure.

FIG. 7 is a top view of a baffle insert in accordance with embodiments of the present disclosure.

FIG. 8 is a top view of a baffle insert in accordance with embodiments of the present disclosure.

FIG. 9 is a top view of a tube configured to receive the baffle insert in accordance with embodiments of the present disclosure.

FIG. 10 is a top view of the baffle insert in accordance with embodiments of the present disclosure outside of the tube in FIG. 9.

FIG. 11 is a top view of the baffle insert in FIG. 10 superimposed over the tube in FIG. 9 to illustrate the different diameters of each (the baffle insert has a larger diameter than the tube).

FIG. 12 is a top view of the baffle insert in FIG. 10 positioned inside the tube in FIG. 9. The interconnection members are deflected and push the baffle members against the interior wall of the tube.

FIG. 13 is a perspective view of a baffle insert in accordance with embodiments of the present disclosure.

### DETAILED DESCRIPTION

A removable baffle insert can be used with a container such as a mini-bioreactor. In some embodiments, the removable baffle insert is configured to fit inside the container and exert a biasing force on the interior wall of the container. The biasing force is sufficient to hold the baffle insert in place while the container is used for various processes involving cell culture growth, microorganism growth, mixing, shaking, inverting the container, and the like.

In some environments, the baffle insert is flexible and has a larger cross-sectional geometry than the that of the container. The baffle insert can be placed in the container by compressing the baffle insert, positioning it inside the container, and allowing it to expand against the interior walls of the container. After the container is used, the baffle insert can be easily removed from the container.

In one potential application, the addition of baffles to a smooth walled container, such as a culture tube, increases the oxygen transfer to the tube's contents, especially into a liquid phase. The increased oxygen transfer facilitates greater biomass growth and more reliable sample results by reducing the likelihood that any observed effect is due to insufficient oxygen transfer.

In containers that are used for mixing, the baffles can be used to disrupt flow and increase mixing. For example, baffles may disrupt the vortex formed in the media when the tube is agitated in a shaker, resulting in turbulent flow within the tube. Turbulent flow may provide greater oxygen flow to the tube's contents than laminar flow. The benefits of baffles are not limited to the use in tubes agitated in a shaker. For example, baffles may increase turbulent flow within a container mixed with a stir bar or manually by a user.

In another potential application, the removable baffle insert can be used to perform a side-by-side biomass production comparison study to isolate the effect of the baffle on the study results. This can be done by conducting a first phase where biomass is produced with the baffle inserts present in the containers and a second phase where biomass is produced without the baffle inserts in the containers. The removable baffle inserts enable such a study to be done for a wide variety of containers.

It should be appreciated that the baffle inserts can be used with a wide variety of containers. For example, other containers that can benefit from the removable baffle insert include reactors, mixers, heat exchangers, and any above ground vessel or piping where the mixing of contents is desired and walls are present.

Although the baffle inserts can be used with any type of container, it is especially suitable for use with bioreactors such as mini bioreactors used in a high throughput screening environment. In these environments, the culture systems can struggle to receive adequate oxygen to support proper cell growth. The addition of the removable baffle insert can be used to increase oxygen transfer in such containers.

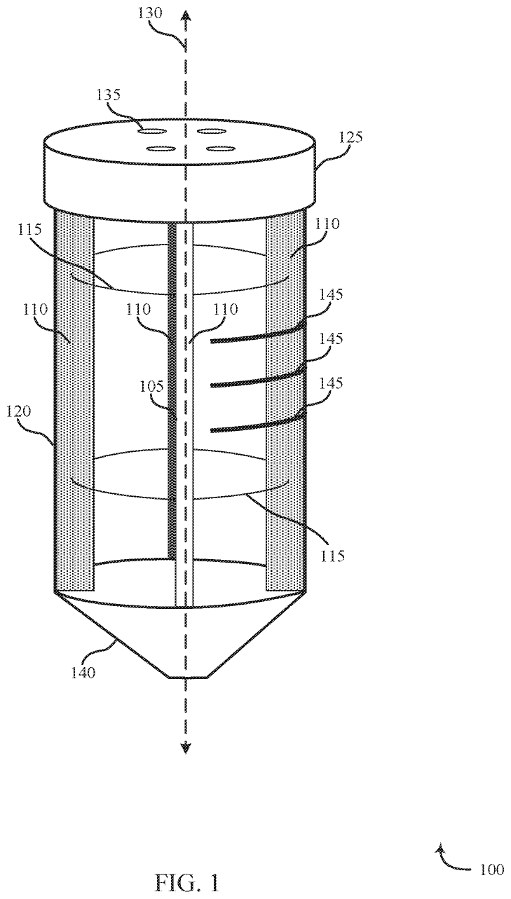

FIG. 1 illustrates an example of a container 100 (alternatively referred to as a bioreactor) that includes a removable baffle insert 105 positioned in a tube 120. The system 100 may also include a cap 125 and have a central longitudinal axis 130.

It should be appreciated that the container 100 can be any suitable size, any suitable shape, and be made of any suitable material. For example, the container 100 can have a volume of approximately 25 ml to approximately 500 ml or approximately 50 ml to approximately 200 ml.

The container 100 can have any suitable shape. For example, the container 100 may have the cylindrical tube shape shown in FIG. 1. In other embodiments, the container 100 can have a square tube shape or rectangular tube shape. The container 100 can also have a conical bottom 140 or, in other embodiments, a flat bottom (alternatively referred to as a bottom end). The conical bottom 140 may be beneficial in that it can better collect the contents in the bottom of the container 100. For example, the conical bottom 140 may allow for more efficient and/or faster centrifugation than a flat bottom tube, and the conical bottom 140 may direct contents, specifically when there is only a small volume of contents, to the bottom of the container 100, which provides increased utility for a user.

The tube 120 can be made of any suitable material. Examples of suitable materials include polymers such as polyolefins, glass, and the like. Materials with no or low reactivity with the anticipated container contents are generally preferred. For example, the tube 120 can be made of low-density polyethylene (LDPE).

In some embodiments, container 100 is a bioreactor. In general, bioreactors are containers (e.g., smooth walled tubes) configured for growing cultures or organisms in an environment where the system inputs and/or outputs are controlled. The cultures may need nutrients and oxygen to grow.

In some embodiments, the container 100 is used for high throughput suspension culture processes. High throughput screening of expression systems and culture conditions may involve high numbers of small volume cultures that are produced in conditions representative of production. The baffle insert 105 can be used to make the screening environment more like the production environment that typically has higher oxygen supply through the use of baffles, sparging, back-pressure, and/or oxygen supplementation. Exemplary high throughput applications of bioreactors may include cell line development, clone selection, media optimization, recombinant protein development, and the like.

The bioreactor can also be sterilized as part of using it to grow cultures and the like. This can be done using heat, radiation (gamma radiation exposure), and the like. The bioreactors can be sterilized to be nonpyrogenic, RNase free, and/or DNase free to avoid any unknown reactants that may lead to unpredicted results.

In some embodiments, the container 100 can be agitated to increase mixing in the media and allow more oxygen to enter the system. Agitation may occur in a shaker for a sustained period of time. The shaker may also include temperature control to optimize reaction conditions.

The cap 125 can be joined or coupled to the top of the tube 120 in any suitable manner. For example, the cap 125 may be threaded to tighten onto internal or external threads of the top of the tube 120. Cap 125 may apply a force on the baffle insert 105 that pushes the baffle members 110 against the top of the conical bottom 140 when the cap 125 is coupled to the tube 120. This may aid in holding the baffle insert 105 in place.

The cap 125 can be made of any suitable material including any of those disclosed above in connection with the tube 120. In some embodiments, the cap 125 and the tube 120 are made of the same material. In other embodiments, the cap 125 and the tube 120 are made of different materials. The cap 125 can include vents 135 that allow oxygen transfer to the contents of the container 100. The presence of oxygen facilitates culture growth in the container 100.

The container 100 can also include markings 145 such as volume markings on the exterior of the tube 120 wall that mark a specific volume (e.g., 10 mL, 20 mL, 30 mL, and the like), which the user may consider when filling and monitoring the volume of the container 100. Although not shown, the container 100 may have an area on the exterior of the tube 120 for labeling it.

The baffle insert 105 can be in a state of radial compression where it is biased against the interior wall of the tube 120. The amount of force can be within a range that allows the baffle insert 105 to remain secured inside the container 100 during various aspects of processing (e.g., mixing, inverting the container 100, and the like) and also be removed from the container 100.

The baffle insert 105 can be configured for a single use application or it can be reusable. The baffle insert 105 can also be manufactured using any suitable process. In some embodiments, the baffle insert 105 is manufactured by injection molding. The baffle insert 105 may include a plurality of elongated baffle members 110 and one or more interconnection members 115.

FIG. 1 shows the baffle insert 105 having four elongated baffle members 110. It should be appreciated, however, that more or fewer baffle members 110 can be present. The baffle members 110 can have a variety of shapes and extend radially inward, towards the central longitudinal axis 130, from the interior of the wall of the tube 120.

It should be appreciated that the baffle members 110 can be made of any suitable material. For example, the baffle members 110 can include polymers such as polyolefins (LDPE), glass, or the like. In general, the baffle members may be made using materials having little or no reactivity with the contents of the container 100. The baffle members 110 can also be made of the same material or different materials than the container 100, the tube 120, and/or the cap 125.

The interconnection members 115 connect and hold the baffle members 110 in place within the container 100. In some embodiments, the interconnection members 115 can be rings that extend around and interconnect with the baffle members 110. The interconnection members 115 can have a spring like flexibility that allows the baffle insert device to transition between different configurations (e.g., flexed within the container 100 and relaxed outside of the container 100).

The modulus of the interconnection members 115 can vary to allow for different biasing forces within the container 100. For example, a higher modulus may be used to exert a higher force on the container 100 wall when it is desired to more securely hold the baffle insert 105 in place and vice versa.

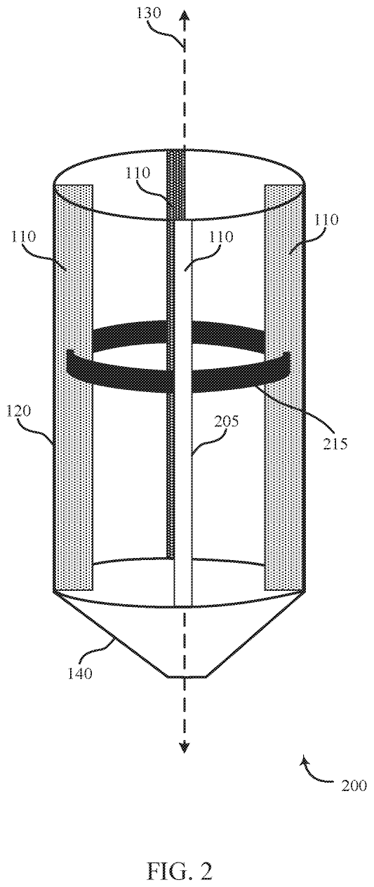

The interconnection members 115 can have any suitable structural configuration. FIGS. 2-4 show examples of suitable shapes and configurations for the interconnection members 115, which include a ring or a bar.

The interconnection members 115 can also be made of any suitable material. In some embodiments, the interconnection members 115 are made of a resilient, flexible material that is capable of flexing and exerting a biasing force on the baffle members 110. Examples of such materials include polymers such as polyolefins, certain metals, and the like. It is generally desirable for the material to be inert and not react with the contents of the container 100. In some embodiments, the one or more interconnection members 115 may be made of the same material as the container 100, the tube 120, the cap 125, and/or the baffle members 110—e.g., low density polyethylene (LDPE), polypropylene (PP), or the like.

In some embodiments, the baffle insert 105 and any of its constituent components—e.g., the baffle members 110, the interconnection members 115, and the like—may be made of a material that is intentionally reactive with the culture material. For example, the baffle insert 105 can be made of or coated or impregnated with a time release material that slowly dissolves and releases nutrients that feed the culture. For example, the baffle insert 105 can be entirely or partly impregnated with glucose that is slowly released to feed the culture. It should be appreciated that any of the baffle inserts described in this document can have this configuration.

FIG. 2 illustrates a perspective view of an embodiment of a container 200 that includes a baffle insert 205 positioned inside the tube 120. The container 200 is similar to the container 100 in FIG. 1 except the baffle insert 205 includes a single, substantially thicker interconnection member 215 than the baffle insert 105 in FIG. 1. It should be appreciated that the baffle insert 205 can include any suitable number of the thicker interconnection members 215. Also, the interconnection member 215 can be flexible and exert a biasing force similar to the interconnection member 115.

The number and cross-sectional thickness of the interconnection members 215 can be adjusted to vary the biasing force of the baffle insert 205 against the inside of the tube 120. For example, a single thick band may have a higher modulus than a single thin ring of the same material. Likewise, more or fewer interconnection members 215 can be used to adjust the amount of biasing force. The material modulus, thickness, and/or number of interconnection members 215 may be tuned to function better for a specific temperature application.

The location where the interconnection members 215 are coupled to the baffle members 110 can vary along the longitudinal length of the baffle members 110. The interconnection members 215 can be positioned in different locations to adjust how and where the force on the baffle members 110 is applied to the inside of the container 100.

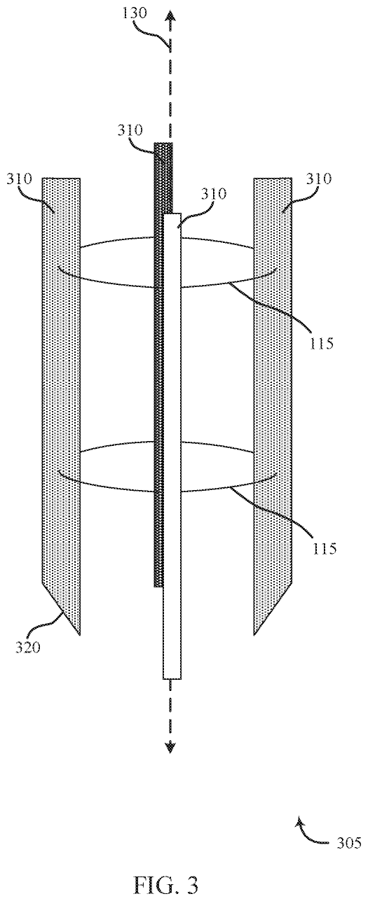

FIG. 3 illustrates a perspective view of an embodiment of a baffle insert 305 having an angled or slanted bottom 320. The angled bottom 320 mirrors the shape of a conical bottom of the tube 120 (e.g., conical bottom 140 in FIGS. 1-2) and may allow for further penetration into the conical bottom of a tube.

The angled bottom 320 can extend inward towards the central longitudinal axis 130 such that the bottom tip of the angled bottom 320 is smaller than the opening of the tube 120. This makes it easier to insert the baffle insert 305 into and out of a container 100 by transitioning the baffle insert device smoothly from a relaxed state to a flexed state and from a flexed state to a relaxed state. In comparison, the baffle members 110, which have a flat bottom may be more difficult to insert into the tube 120.

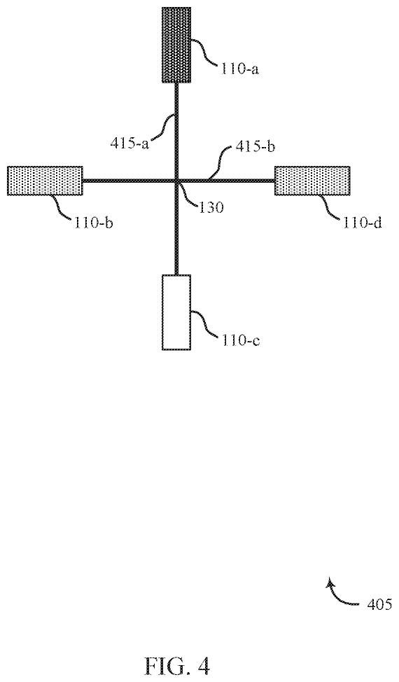

FIG. 4 illustrates a top view of an embodiment of a baffle insert 405. The baffle insert 405 is similar to the other baffle inserts shown in FIGS. 1-3 in that it can be configured to fit inside the container 100 and bias the baffle member against the interior wall of the container 100. However, the baffle insert 405 includes baffle members 110 that are coupled together by interconnection members 415 that extend cross-wise in the tube 120 between opposing baffle members 110. The interconnection members 415 meet in the center at the longitudinal axis 130 of the container 100.

In this configuration, the interconnection members 415 can be seen as cross bars that connect at the central longitudinal axis 130. The central connection may be near the opening of a container 100, and a cross bar or the central connection may be used as a handle to insert and remove the baffle insert 405 into and out of the container 100. For example, using a pipet to push the baffle insert 405 out of a container opening. Pulling on the central connection of flexible interconnection members 415 may pull the baffle members 110 inward and debiasing them from the side wall of the tube 120 making extraction easier.

The interconnection members 415 connect and hold the baffle members 110 in place in the container 100. The interconnection member 415-a connects the baffle members 110-a, 110-c, and the interconnection member 415-b connects the baffle members 110-b, 110-d. The baffle members 110 are shaded to show the relationship between a top view of a baffle insert 405 and a perspective view as shown in FIGS. 1-3 and 10.

The interconnection members 415 can be made of a spring like material that is flexible and resilient to allow the baffle insert 405 to transition between the different configurations (e.g., compressed within the container 100 and relaxed outside of the container 100). When the baffle insert 405 is compressed in the container 100, the interconnection members 415 can be compressed towards the central longitudinal axis 130 and/or deflected sideways.

For example, the interconnection member 415-a can be linearly compressed and bias the outer edge of the baffle members 110-a and 110-c against the interior wall of the container 100. The modulus of the one or more interconnection members 415 can vary to allow for different biasing forces within the container 100. For example, a higher modulus may be used to exert a higher force on the interior wall of the container 100.

FIG. 5 illustrates a top view of an embodiment of a baffle insert 505. The baffle members 110 have a rectangular cross section and are coupled to one or more interconnection members 115 at approximately the middle of each baffle member 110. The baffle members 110 extend parallel to the central longitudinal axis 130. The inner edge of the elongated baffle members 110 is the edge closest to the central longitudinal axis 130, and the outer edge of the elongated baffle members 110 is the edge furthest from the central longitudinal axis 130. The outer edge contacts the inner wall of the container 100. The middle of each elongated baffle member 110 is equidistant from the inner edge and the outer edge of each elongated baffle member 110.

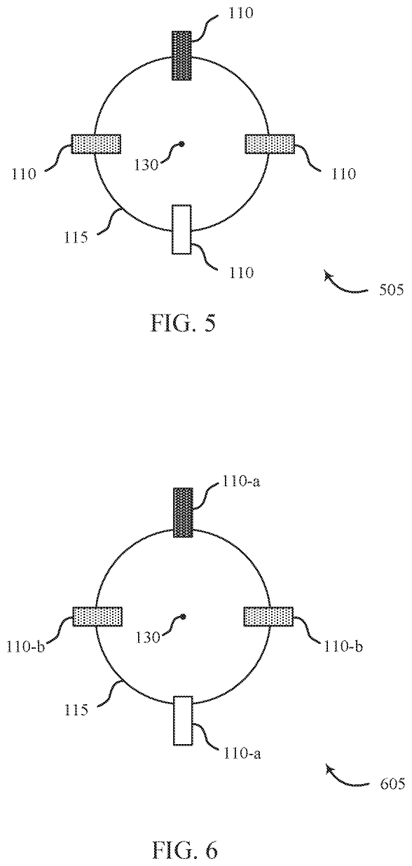

FIG. 6 illustrates a top view of an embodiment of a baffle insert 605 that is similar to the baffle insert 505. The baffle insert 605 differs from the baffle insert 505 in that the interconnection member 115 is coupled to the baffle members 110-a at a location that is closer to the inner edge than the outer edge. The interconnection member 115 is coupled to the baffle members 110-b at the same location in both the baffle inserts 505, 605. In this configuration, the baffle members 110-a exert a greater amount of force against the interior of the container 100 than the baffle members 110-b. Adjustments such as these can be used to change the amount of force exerted by each baffle member 110 against the interior wall of the container 100.

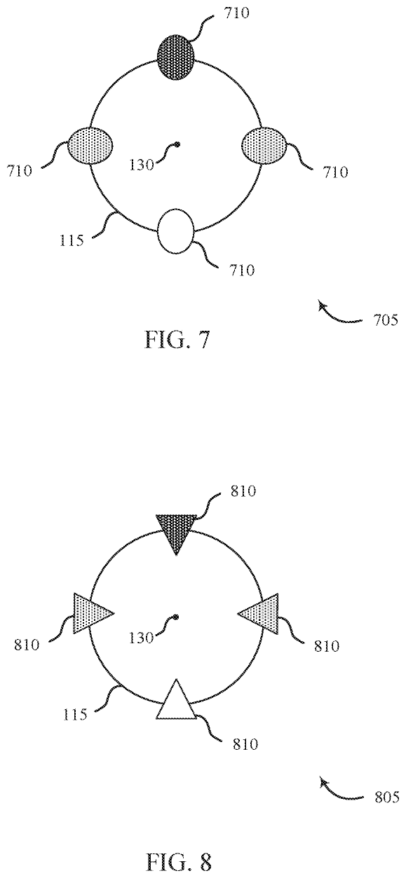

FIGS. 7-8 show cross-sectional views of embodiments of baffle inserts 705, 805 where the baffle members 710, 810 have various cross-sectional shapes. The baffle members 710 have a circular or oval cross section. The baffle members 810 have a triangular cross section. It should be appreciated that the baffle members can have numerous other shapes besides those shown in FIGS. 7-8.

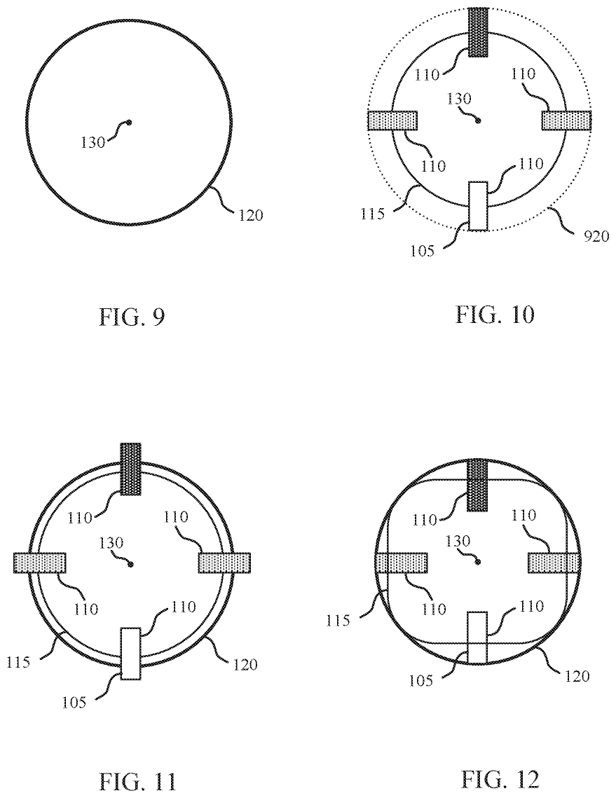

FIGS. 9-12 show how the baffle insert 105 fits inside the container 100. FIG. 9 shows the cross-sectional area (e.g., circumference) of the container 100. The central longitudinal axis 130 is shown inside the container 100. It should be noted that although the container 100 is depicted as a cylinder, it can have any other suitable cross-sectional shape.

FIG. 10 shows the cross-sectional area of the baffle insert 105. The cross-sectional area is shown by a circle 920 that extends around the outer edge of the elongated baffle members 110. FIG. 11 shows the cross-sectional area of the baffle insert 105 overlaid on the cross-sectional area of the container 120. This is the cross-sectional area of the baffle insert 105 in the relaxed state. As illustrated, the cross-sectional area of the baffle insert 105, in the relaxed state and around the outer edge of the elongated baffle members 110, is larger than the cross-sectional area of the container 120.

FIG. 12 shows the baffle insert 105 positioned in the container 100. The baffle insert 105 is in a flexed state. In this state, the one or more interconnection members 115 deflect in a spring-like manner so that the interconnection members 115 are compressed. In this flexed state, the interconnection members 115 exert an outward force on the baffle members 110 and bias them against the inside of the container 100.

It should be appreciated that the cross-sectional area of the baffle insert 105 may be the same or even slightly smaller than the cross-sectional area of the interior wall of container 100.

The removable baffle insert 105 may be arranged in container 100 such that the container may undergo mixing (e.g., torsional force) while the baffle insert 105 remains stationary in a biased position against the interior container wall relative to the contents of the container 100.

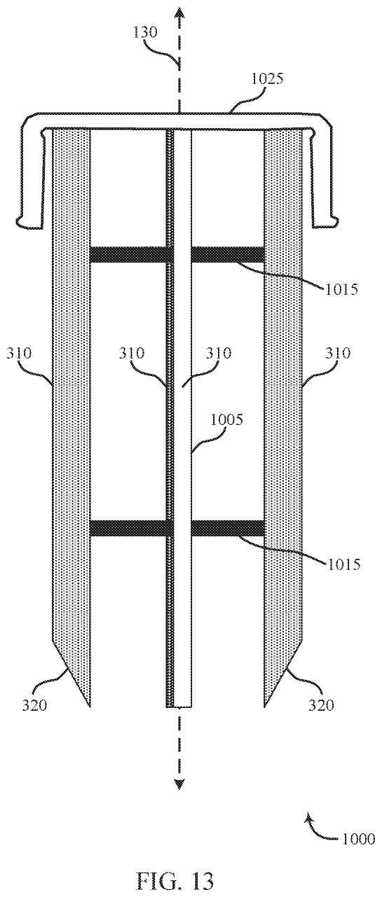

FIG. 13 illustrates a perspective view of an embodiment of a baffle insert 1000. In this embodiment, the baffle members 310 are coupled to the underside of the cap 1025 such that when the cap is joined or coupled to the container 100 the baffle insert 1000 is secured within the container 100.

It should be appreciated that the baffle insert 1000 can be coupled to the underside of the cap 1025 using any suitable technique. In some embodiments, the baffle insert 1000 is formed as an integral component of the cap 1025. In some other embodiments, the baffle insert 1000 is a separate component that is attached to the underside of the cap 1025 using adhesive or the like.

The cap 1025 may be threaded such that it tightens onto internal or external threads on the top of the container 100. In some embodiments, the cap 1025 applies a force on the baffle insert 1000 that pushes the baffle members 310 against the bottom and/or sides of the container 100 when the cap 1025 is coupled to the container 100. This can aid in holding the baffle insert 1005 in place. In other embodiments, the baffle insert 105 can extend into the container without touching the bottom and/or sides of the container 100.

The cap 1025 can be made of a polymer such as a polyolefin, glass, or the like. Materials with no or low reactivity with container contents may be preferred. For example, the cap 1025 may be made of LDPE. The cap 1025 can be made of the same or different material as the baffle members 310. The cap 125 can also include vents, not shown, that allow oxygen transfer to the contents of the container.

FIG. 10 shows four elongated baffle members 310. However, it should be appreciated that more or fewer baffle members 310 can be present. The baffle members 310 can be formed in a variety of shapes and extend radially inward, towards the central longitudinal axis 130.

The baffle insert device 1000 can include one or more interconnection members 1015. The one or more interconnection members 1015 may be thick bands as shown in FIG. 2, or they can be another shape such as rings or cross bars. The baffle members 310 and one or more interconnection members 1015 may be chosen from the variety of examples provided in relation to FIGS. 1-8.

Terminology and Interpretative Conventions

The term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate member being attached to one another. Such joining may be permanent in nature or alternatively may be removable or releasable in nature.

The term “coupled” includes joining that is permanent in nature or releasable and/or removable in nature. Permanent joining refers to joining the components together in a manner that is not capable of being reversed or returned to the original condition. Releasable joining refers to joining the components together in a manner that is capable of being reversed or returned to the original condition.

Releasable joining can be further categorized based on the difficulty of releasing the components and/or whether the components are released as part of their ordinary operation and/or use. Readily or easily releasable joining refers to joining that can be readily, easily, and/or promptly released with little or no difficulty or effort. Difficult or hard to release joining refers to joining that is difficult, hard, or arduous to release and/or requires substantial effort to release. The joining can be released or intended to be released as part of the ordinary operation and/or use of the components or only in extraordinary situations and/or circumstances. In the latter case, the joining can be intended to remain joined for a long, indefinite period until the extraordinary circumstances arise.

It should be appreciated that the components can be joined together using any type of fastening method and/or fastener. The fastening method refers to the way the components are joined. A fastener is generally a separate component used in a mechanical fastening method to mechanically join the components together. A list of examples of fastening methods and/or fasteners are given below. The list is divided according to whether the fastening method and/or fastener is generally permanent, readily released, or difficult to release.

Examples of permanent fastening methods include welding, soldering, brazing, crimping, riveting, stapling, stitching, some types of nailing, some types of adhering, and some types of cementing. Examples of permanent fasteners include some types of nails, some types of dowel pins, most types of rivets, most types of staples, stitches, most types of structural ties, and toggle bolts.

Examples of readily releasable fastening methods include clamping, pinning, clipping, latching, clasping, buttoning, zipping, buckling, and tying. Examples of readily releasable fasteners include snap fasteners, retainer rings, circlips, split pin, linchpins, R-pins, clevis fasteners, cotter pins, latches, hook and loop fasteners (VELCRO), hook and eye fasteners, push pins, clips, clasps, clamps, zip ties, zippers, buttons, buckles, split pin fasteners, and/or conformat fasteners.

Examples of difficult to release fastening methods include bolting, screwing, most types of threaded fastening, and some types of nailing. Examples of difficult to release fasteners include bolts, screws, most types of threaded fasteners, some types of nails, some types of dowel pins, a few types of rivets, a few types of structural ties.

It should be appreciated that the fastening methods and fasteners are categorized above based on their most common configurations and/or applications. The fastening methods and fasteners can fall into other categories or multiple categories depending on their specific configurations and/or applications. For example, rope, string, wire, cable, chain, and the like can be permanent, readily releasable, or difficult to release depending on the application.

Certain aspects of one or more embodiments of this disclosure are provided below.

Aspect (1) pertains to a baffle insert for a tube. The baffle insert includes a plurality of elongated baffle members, the plurality of elongated baffle members being able to fit in the tube and contact an interior wall of the tube. The baffle inserts also includes an interconnection member coupled to the plurality of elongated baffle members, the interconnection member holding the plurality of elongated baffle members together in a spaced apart relationship.The baffle insert has a cross-sectional geometry that is larger than a cross-sectional geometry of the tube, and the interconnection member is configured to bias the plurality of baffle members against the interior wall of the tube.

Aspect (2) pertains to the baffle insert of Aspect (1), where the interconnection member is flexible.

Aspect (3) pertains to the baffle insert of Aspect (1) or Aspect (2), wherein the interconnection member is able to move between a relaxed state when the baffle insert is not positioned in the tube and a flexed state when the baffle insert is positioned in the tube and the interconnection member biases the plurality of baffle members against the interior wall of the tube.

Aspect (4) pertains to the baffle insert of any one of Aspects (1)-(3), wherein the interconnection member is arranged perpendicular to the plurality of baffle members.

Aspect (5) pertains to the baffle insert of any one of Aspects (1)-(4), wherein the plurality of baffle members can extend parallel to a longitudinal axis of the tube when the baffle insert is positioned in the tube.

Aspect (6) pertains to the baffle insert of any one of Aspects (1)-(5), wherein the interconnection member comprises a flexible band.

Aspect (7) pertains to the baffle insert of any one of Aspects (1)-(6), wherein the interconnection member comprises a crossbar.

Aspect (8) pertains to the baffle insert of any one of Aspects (1)-(7), wherein a composition of the plurality of baffle members includes a polyolefin.

Aspect (9) pertains to the baffle insert of any one of Aspects (1)-(8), wherein the baffle insert has a diameter that is larger than a diameter of the tube.

Aspect (10) pertains to a bioreactor including: a tube including an opening at a top end of the tube; and a removable baffle insert positioned in the tube. The baffle insert includes a plurality of baffle members coupled together by an interconnection member. The bioreactor is configured to facilitate the growth of microorganisms in the tube and on the baffle insert.

Aspect (11) pertains to the bioreactor of Aspect (10), wherein the plurality of baffle members includes angled bottoms.

Aspect (12) pertains to the bioreactor of Aspect (10) or Aspect (11), wherein the tube includes a conical bottom.

Aspect (13) pertains to the bioreactor of any one of Aspects (10)-(12), wherein the bioreactor includes a culture of cells and/or microorganisms in the tube.

Aspect (14) pertains to the bioreactor of any one of Aspects (10)-(13), further including a cap coupled to the opening of the tube.

Aspect (15) pertains to the bioreactor of Aspect (14), wherein the baffle insert is coupled to the cap.

Aspect (16) pertains to the bioreactor of any one of Aspects (10)-(15), wherein a composition of the tube includes a polyolefin or glass.

Aspect (17) pertains to a method of culturing cells and/or microorganisms using the bioreactor of any one of Aspects (10)-(16).

Aspect (18) pertains to a container including: a tube including an opening at a top end of the tube; and a baffle insert positioned in the tube, the baffle insert including a plurality of baffle members coupled together by an interconnection member. The interconnection member biases the plurality of baffle members against an interior wall of the tube.

Aspect (19) pertains to the container of Aspect (18), wherein the baffle insert includes a cross-sectional geometry when it is not positioned in the tube that is larger than a cross-sectional geometry of the tube.

Aspect (20) pertains to the container of Aspect (18) or (19), wherein the container is a bioreactor.

Aspect (21) pertains to a container including: a tube including an opening at a top end of the tube; a cap coupled to the top end of the tube; and a baffle insert positioned in the tube, the baffle insert including a plurality of baffle members coupled together by an interconnection member. The cap and the baffle insert are in contact with each other and the cap holds the baffle insert in place in the tube.

Aspect (22) pertains to the container of Aspect (21), wherein the baffle insert is coupled to the cap.

Aspect (23) pertains to the container of Aspect (21), wherein the cap and the baffle insert are integral components.

Aspect (24) pertains to the container of any one of Aspects (21)-(23), wherein the tube includes a bottom end, and wherein the cap biases the baffle insert against the bottom end of the tube.

Any methods described in the claims or specification should not be interpreted to require the steps to be performed in a specific order unless stated otherwise. Also, the methods should be interpreted to provide support to perform the recited steps in any order unless stated otherwise.

Spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting.

Articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all the items together, or any combination or number of the items.

The terms have, having, include, and including should be interpreted to be synonymous with the terms comprise and comprising. The use of these terms should also be understood as disclosing and providing support for narrower alternative embodiments where these terms are replaced by “consisting” or “consisting essentially of.”

Unless otherwise indicated, all numbers or expressions, such as those expressing dimensions, physical characteristics, and the like, used in the specification (other than the claims) are understood to be modified in all instances by the term “approximately.” At the very least, and not as an attempt to limit the application of the doctrine of equivalents to the claims, each numerical parameter recited in the specification or claims which is modified by the term “approximately” should be construed in light of the number of recited significant digits and by applying ordinary rounding techniques.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

All disclosed numerical values are to be understood as being variable from 0-100% in either direction and thus provide support for claims that recite such values or any and all ranges or subranges that can be formed by such values. For example, a stated numerical value of 8 should be understood to vary from 0 to 16 (100% in either direction) and provide support for claims that recite the range itself (e.g., 0 to 16), any subrange within the range (e.g., 2 to 12.5) or any individual value within that range (e.g., 15.2).

The drawings shall be interpreted as illustrating one or more embodiments that are drawn to scale and/or one or more embodiments that are not drawn to scale. This means the drawings can be interpreted, for example, as showing: (a) everything drawn to scale, (b) nothing drawn to scale, or (c) one or more features drawn to scale and one or more features not drawn to scale. Accordingly, the drawings can serve to provide support to recite the sizes, proportions, and/or other dimensions of any of the illustrated features either alone or relative to each other. Furthermore, all such sizes, proportions, and/or other dimensions are to be understood as being variable from 0-100% in either direction and thus provide support for claims that recite such values or any and all ranges or subranges that can be formed by such values.

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “invention,” etc., are not meant to invoke exception (b) or otherwise restrict the scope of the recited claim terms. Other than situations where exception (b) applies, nothing contained in this document should be considered a disclaimer or disavowal of claim scope.

The subject matter recited in the claims is not coextensive with and should not be interpreted to be coextensive with any embodiment, feature, or combination of features described or illustrated in this document. This is true even if only a single embodiment of the feature or combination of features is illustrated and described in this document.

1. A baffle insert for a tube comprising:
a plurality of elongated baffle members, the plurality of elongated baffle members being configured to fit in the tube and contact an interior wall of the tube; and
an interconnection member coupled to the plurality of elongated baffle members, the interconnection member holding the plurality of elongated baffle members together in a spaced apart relationship;
wherein the baffle insert has a cross-sectional geometry that is larger than a cross-sectional geometry of the tube; and
wherein the interconnection member is configured to bias the plurality of baffle members against the interior wall of the tube.

2. The baffle insert of claim 1, wherein the interconnection member is flexible.

3. The baffle insert of claim 1, wherein the interconnection member is configured to move between a relaxed state when the baffle insert is not positioned in the tube and a flexed state when the baffle insert is positioned in the tube and the interconnection member biases the plurality of baffle members against the interior wall of the tube.

4. The baffle insert of claim 1, wherein the interconnection member is arranged perpendicular to the plurality of baffle members.

5. The baffle insert of claim 1, wherein the plurality of baffle members are configured to extend parallel to a longitudinal axis of the tube when the baffle insert is positioned in the tube.

6. The baffle insert of claim 1, wherein the interconnection member comprises a flexible band.

7. The baffle insert of claim 1, wherein the interconnection member comprises a crossbar.

8. The baffle insert of claim 1, wherein a composition of the plurality of baffle members comprises a polyolefin.

9. The baffle insert of claim 1, wherein the baffle insert has a diameter that is larger than a diameter of the tube.

10. (canceled)

11. (canceled)

12. (canceled)

13. (canceled)

14. (canceled)

15. (canceled)

16. (canceled)

17. (canceled)

18. A container comprising:
a tube including an opening at a top end of the tube; and
a removable baffle insert positioned in the tube, the baffle insert comprising a plurality of baffle members coupled together by an interconnection member;
wherein the interconnection member biases the plurality of baffle members against an interior wall of the tube.

19. The container of claim 18, wherein the baffle insert comprises a cross-sectional geometry when it is not positioned in the tube that is larger than a cross-sectional geometry of the tube.

20. The container of claim 18, wherein the container is a bioreactor configured to facilitate the growth of microorganisms in the tube.

21. (canceled)

22. (canceled)

23. (canceled)

24. (canceled)

25. The container of claim 18, wherein the tube comprises a conical bottom.

26. The container of claim 25, wherein the plurality of baffle members comprises angled bottoms.

27. The container of claim 18, further comprising a cap coupled to the opening of the tube.

28. The container of claim 27, wherein the baffle insert is coupled to the cap.

29. The container of claim 27, wherein the cap and the baffle insert are in contact with each other and the cap holds the baffle insert in place in the tube.

30. The container of claim 27, wherein the tube includes a bottom end, and wherein the cap biases the baffle insert against the bottom end of the tube.

31. A method of culturing cells and/or microorganisms using the container of claim 18.
